(19) United States
(12) Patent Application Publication
(10) Pub. No.: US 2025/0236576 A1
(43) Pub. Date: Jul. 24, 2025

(54) PROCESS

(71) Applicant: GIVAUDAN SA, VERNIER (CH)

(72) Inventors: Corinne BAUMGARTNER, Kemptthal (CH); Michael GEORGE, Nottingham (GB); Martyn POLIAKOFF, Nottingham (GB); Fridtjof SCHROEDER, Kemptthal (CH); Bryan TAMBOSCO, Nottingham (GB)

(21) Appl. No.: 18/842,144

(22) PCT Filed: Mar. 15, 2023
(86) PCT No.: PCT/EP2023/056561

§ 371 Date: Aug. 28, 2024

(30) Foreign Application Priority Data
Mar. 17, 2022 (GB) 2203697.4

(51) Int. Cl. C07C 29/50; C07D 309/04

(52) U.S. Cl. CPC C07C 29/50; C07D 309/04; C07B 2200/07

(57) ABSTRACT

Described herein are methods for making intermediates useful in the production of fragrances. In particular, a photooxidation method is disclosed wherein the photooxidation takes place in the present of molecular oxygen and an aromatic solvent comprising a sensitizer.

### TECHNICAL FIELD

The present disclosure refers to fragrance ingredients or intermediates thereof and to methods of forming the same. In particular, the present disclosure relates to a method for making diols which are suitable for the production of fragrances, such as Rose Oxide.

### BACKGROUND

Rose Oxide (2-(2-methylprop-1-enyl)-4-methyltetrahydropyran) is a well-known and desirable fragrance material, used in cosmetic products and detergents. There are a number of different methods of producing this material, one of them being the acid catalyzed cyclisation of 3,7-dimethyloct-5-ene-1,7-diol and/or 3,7-dimethyloct-7-ene-1,6-diol (U.S. Pat. No. 5,892,059). The diols are generally available, for example, by photooxidation of citronellol. To initiate the photooxidation, for example, rose bengal in methanol (GB1,010,056), or tetraphenylporphyrin (TPP) in methanol, ethanol, dichloromethane or trichloromethane are described in the literature (see, for example, C. Horn, S. Gremetz, BeilJOC 2020; A. Favre-Reguillon et al. ChemPhotoChem 2019; and F. Lévesque, P. H. Seeberger, Org Lett 2011).

In all the methods described, however only relative low substrate concentrations of 0.1 to 0.5 M have been used in the presence of about 0.1 to 0.25 mM of a sensitizer, resulting in a high conversion rate. For a process to be cost-efficient on an industrial scale, it is however desirable to be very productive. So in order to increase productivity, it is desirable to use a process which is efficient even at higher substrate concentration. Furthermore, it would be desirable to obtain a product that can be used directly as an intermediate product in a subsequent process step without purification.

It is therefore desirable to provide a new efficient access to obtain a diol mixture which can be used in the production of fragrance ingredients, such as Rose Oxide, without separation of the diols from the solvent present in the reaction mixture.

### SUMMARY

In accordance with a first aspect of the present invention, there is provided a method for making a mixture comprising a compound of Formula (II), a compound of Formula (III), and, if R¹ is not methyl, a compound of Formula (V)

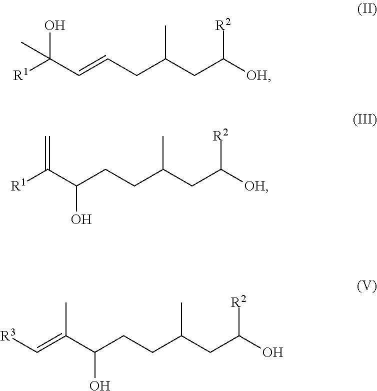

wherein the method comprises photooxidation of a compound of Formula (I) in an aromatic solvent comprising a sensitizer in the presence of molecular oxygen,

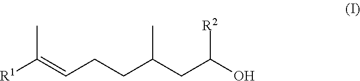

wherein
R¹ is selected from C₁-C₅ alkyl (e.g., methyl, ethyl, propyl, iso-propyl),
R² is selected from hydrogen and C₁-C₄ alkyl (e.g., methyl, ethyl, propyl, iso-propyl),
R³ is selected from C₁-C₄ alkyl (e.g., methyl, ethyl, propyl, iso-propyl); and
wherein the sensitizer is a porphyrin.

In a second aspect of the present invention, there is provided a method according to the first aspect followed by cyclisation to a compound of Formula (IV) in the presence of an acid

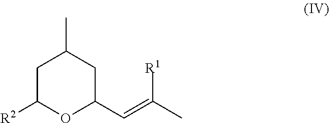

wherein R¹ and R² have the same meaning as defined in the first aspect.

### DETAILED DESCRIPTION

The present invention provides a novel and surprising method for the production of a mixture comprising a compound of Formula (II) and a compound of Formula (III), which may be used for the production of compounds of Formula (IV), e.g. Rose Oxide (R¹=CH₃ and R²=H), obtainable by cyclisation in the presence of an acid without the requirement to change the solvent.

In particular, the present invention is based, at least in part, on the surprising finding that photooxidation of a compound of Formula (I) in an aromatic solvent containing a porphyrin is very efficient. Conversion rates of 95% or higher have been observed, not only at substrate concentrations of around 0.5 M, but also at higher substrate concentrations, such as 1 M or higher (e.g. up to 3M, such as 0.75 M to 2.5M, or 1 M to 2M).

Thus there is provided in one particular embodiment a method for making a mixture comprising a compound of Formula (II), a compound of Formula (III), and, if R¹ is not methyl, optionally a compound of Formula (V)

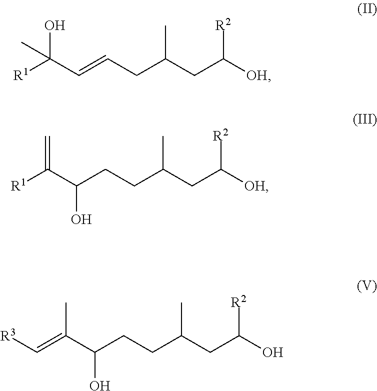

wherein the method comprises photooxidation of a compound of Formula (I) in an aromatic solvent comprising a sensitizer in the presence of molecular oxygen,

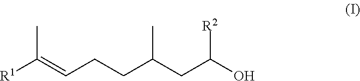

wherein
R¹ is selected from C₁-C₅ alkyl (e.g, methyl, ethyl, propyl, iso-propyl),
R² is selected from hydrogen and C₁-C₄ alkyl (e.g., methyl, ethyl, propyl, iso-propyl),
R³ is selected from C₁-C₄ alkyl (e.g., methyl, ethyl, propyl, iso-propyl); and
wherein the sensitizer is a porphyrin.

In one particular embodiment there is provided a method for making a mixture comprising or consisting of (E)-3,7-dimethyloct-5-ene-1,7-diol and 3,7-dimethyloct-7-ene-1,6-diol, wherein the method comprises photooxidation of citronellol (i.e. a compound of Formula (I) wherein R¹ is methyl and R² is H) in an aromatic solvent comprising a sensitizer in the presence of molecular oxygen.

Citronellol (also known under the name dihydrogeraniol), is a natural acyclic monoterpenoid. Both enantiomers occur in nature. (+)-Citronellol, which is found in citronella oils, including Cymbopogon nardus (50%), is the more common isomer. (−)-Citronellol is found in the oils of rose (18-55%) and Pelargonium geraniums. However, both enantiomers of citronellol can also be prepared by asymmetric catalytic hydrogenation of geraniol or nerol. Or else, it may, for example, be made from renewable myrcene, which is accessible from renewable Pinene. Alternatively it could be made from Citral, which is accessible from synthetic or renewable isobutylene.

In another embodiment there is provided a method for making a mixture comprising or consisting of (E)-3,7-dimethylnon-5-ene-1,7-diol (a compound of formula (II) wherein R¹ is ethyl and R² is H), (E)-3,7-dimethylnon-7-ene-1,6-diol (a compound of formula (V) wherein R² is H and R³ is methyl, and 3-methyl-7-methylenenonane-1,6-diol (compound of formula (III) wherein R¹ is ethyl and R² is H), wherein the method comprises photooxidation of ethylcitronellol (i.e. a compound of formula (I) wherein R¹ is ethyl and R² is H) in an aromatic solvent comprising a sensitizer in the presence of molecular oxygen.

In one particular embodiment ethylcitronellol may be obtained from renewable building blocks.

To initiate the photooxidation a sensitizer is required. The use of porphyrin in an aromatic solvent has been proven as the perfect sensitizer.

Suitable porphyrins are porphyrins of Formula (A) or a metal complex thereof (Formula (B)) wherein the metal (M) is a transition metal (II) (such as Pd(II) and Zn(II)) or MX(III) wherein X is selected from halides (e.g. Cl) (for example, MX(III) is Ga(III) CI),

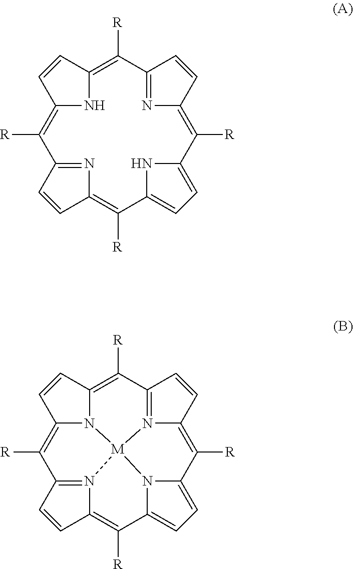

wherein R is selected from phenyl, and substituted phenyl (e.g., para-toluyl, 4-chlorophenyl, 4-trifluorophenyl, 4-bromophenyl, 2,6-difluorophenyl, 2,6-dichlorophenyl, and pentafluorophenyl).

5 By “transition metal” we mean any element in the d-block of the periodic table, such as palladium and zinc.

As specific examples one may mention tetraphenylporphyrin (CAS 917-23-7); gallium(III) 5,10,15,20-(tetraphenyl) porphyrin chloride (CAS 8833-52-0); palladium (II) 5,10,15,20-(tetraphenyl)porphyrin (CAS 14187-13-4); zinc (II) 5,10,15,20-(tetraphenyl)porphyrin (CAS 14074-80-7); 5,10,15,20-(tetra-p-tolyl)porphyrin (CAS 14527-51-6); 5,10,15,20-(tetra-4-chlorophenyl)porphyrin (CAS 22112-77-2); 5,10,15,20-(tetra-4-trifluoromethylphenyl)-porphyrin (CAS 56420-24-7); 5, 10,15,20-(tetra-4-bromophenyl)porphyrin (CAS 29162-73-0); 5,10,15,20-(tetra-2,6-difluorophenyl)porphyrin (CAS 104322-39-6); 5,10,15,20-(tetra-2,6-chlorophenyl)porphyrin (CAS 37083-37-7); 5,10,15,20-(tetrapentafluorophenyl)porphyrin (CAS 25440-14-6); Zinc complex of 5,10,15,20-(tetra-4-chlorophenyl)porphyrin (CAS 29116-33-4); and Zinc complex of 5,10,15,20-(tetra-4-bromophenyl)porphyrin (CAS 29116-34-5). In one particular embodiment the porphyrin is tetraphenylporphyrin (TPP).

Almost complete conversion was already observed at sensitizer concentrations of about 0.1 mol %. Thus there is provided in a further embodiment a method according to the first aspect wherein the sensitizer is present at a concentration of 0.1 mol % or less (e.g. 0.09 mol %, 0.08 mol %, 0.07 mol %, 0.06 mol %, 0.05 mol %, 0.04 mol %, 0.03 mol %, 0.02 mol %, 0.01 mol %, or less).

The use of an aromatic solvent results in a very efficient conversion of the substrate. The aromatic solvent may be selected from toluene, xylene, and chlorobenzene. In one particular embodiment the aromatic solvent is toluene.

The photooxidation takes place in the presence of molecular oxygen. The oxygen present in the air is usually sufficient but pure oxygen or mixtures of oxygen and nitrogen can be also used. The higher the concentrations of oxygen are required it might be an advantage to use pure oxygen rather than air, purely because of the volumes of gas needed for a stoichiometry of the reaction. The reaction mixture is exposed to a source of artificial or natural radiation which can emit light which does not have any short-wave ultra-violet component, more particular light in the range from about 350 to 750 nm, for example 400-700 nm. In one particular embodiment the photooxidation is carried out using an LED (light emitting diode) as the light emitting source.

In one particular embodiment, the amount of light (luminous flux measured in lumen), is in the range of 15000 to 100000 lumens generated by LED's (light emitting diodes) which consist of metal blocks fitted with chips and cooled down using a cooling system. Typically, LED chips are made of a semi-conductor emitting in the blue area of the visible spectrum (around 450 nm) coated with a layer of light emitting phosphor, absorbing some of the blue light emitted by the semi-conductor and phosphorescent visible light around 600 nm. Those chips are also called phosphor-converted white LED's. The combination of the two allows perceiving the emitted light as white.

The method described hereinabove may take place in batch or in continues flow. If a continues flow reactor is used, any form of reactor that is set-up for continuous flow conditions are suitable. Continuous flow reactors, such as PFA tubings around a corn lamp (Favre-Reguillon et al. ChemPhotoChem 3, 122-128, 2019), a Corning reactor (G. Gauron et al. Chemistry Today 36, 12-15, 2018 and L. Dreesen, J.-C. M. Monbaliu et al. OPRD 21, 1435-1438, 2017), the Vortex reactor (M. Poliakoff, M. W. George et al. OPRD 21, 1042-1050, 2017), a gas-liquid membrane reactor (A. Kouridaki, K. Huvaere Reaction Chemistry & Engineering 2, 590, 2017), monochannel microreactors (C. P. Park et al. RSC Advances 5, 4233, 2015), triple-channel microreactors (D.-P. Kim et al, Lab Chip 11, 1941, 2011), a tubular sapphire reactor (M. Poliakoff, M. W. George Angew. Chem. Int. Ed. 48, 5322, 2009), a LTF microreactor (S. Meyer et al. J. Photochem & Photobiology 186, 248, 2007) or the Booker-Milburn reactor (K. I. Booker Milburn et al. J. org. Chem. 70, 7558, 2005) can be used or any related set-up where LED's or mercury pressure lamps or other light sources emit light into a flow of substrate to be oxidized.

The diol mixture obtained according to the first aspect of the invention might be used as an intermediate for the production of fragrance ingredients, such as Rose Oxide or 4-methyl-2-(2-methylbut-1-en-1-yl) tetrahydro-2H-pyran (Ethyl Rose Oxide), without separating the diol mixture from the used aromatic solvent.

Thus, there is provided in a second aspect of the present invention, a method according to the first aspect followed by cyclisation of the diol(s) of Formula (II), (III) and (V) to a compound of Formula (IV) in the presence of an acid

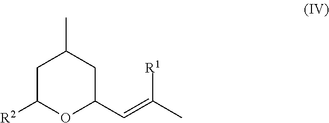

wherein R¹ and R² have the same meaning as defined in the first aspect.

In one particular embodiment the compound of Formula (IV) is enriched in the cis-isomer (IVa)

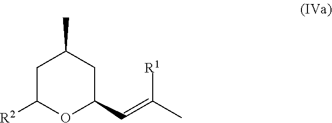

wherein R¹ and R² have the same meaning as defined in the first aspect.

In another particular embodiment the compound of Formula (IV) is enriched in the cis-isomer in the ratio from 55:45 or greater (e.g., in a ratio of 60:40, 70:30, 80:40, 90:10, 95:5 or greater of cis-isomer (IVa): trans-isomer).

Acidic cyclisation of diols of Formula (II), optionally comprising a diol of Formula (III) and a diol of Formula (V) is well known to the skilled person. The term “acid” refers to inorganic acids such as sulfuric acid, H₃PO₄, and HCl (all of which are aqueous solutions), and sulfonic acids such as para-toluene sulfonic acid, methanesulfonic acid, and camphersulfonic acid. All acids may be used with or without phase transfer catalysts such as Bu₄NCl, Bu₄NBr and Aliquat. All acids may be used with or without organic co-solvents such as hexane, heptane, cyclohexane, toluene, xylene. Reaction temperatures are ambient temperature to reflux.

The invention is now further described with reference to the following non-limiting examples. These examples are for the purpose of illustration only and it is understood that variations and modifications can be made by one skilled in the art.

### General

GCMS: 50° C./2 min, 20° C./min 240° C., 35° C./min 270° C. Agilent 5975C MSD with HP 7890A Series GC system. Non-polar column: BPX5 from SGE, 5% phenyl 95% dimethylpolysiloxan 0.2 mm×0.25 μm×12 m. Carrier gas: helium. Injector temperature: 230° C. Split 1:50. Flow: 1.0 ml/min. Transfer line: 250° C. MS-quadrupol: 160° C. MS-source: 230° C. Injection vol. 1 μl. Ionization mode Electron Impact (El) at 70 eV.

GC: 100° C./2 min, 15° C./min 240° C., 240° C./5 min. Thermo Focus GC. Non-polar column: Agilent Technologies J&W Scientific DB-5 ((5% Phenyl)-methylpolysiloxane) 0.32 mm×0.25 μm×30 m. Carrier gas: helium. FID-Detector, Detector temp. 270° C. Injector temperature: 240° C. Split 1:42.3. Pressure 70 kPa.

¹H- and ¹³C-NMR: Bruker-DPX-400 MHz spectrometer; spectra were recorded at 400 MHZ (¹H) and 100 MHz (¹³C) respectively in CDCl₃; δ in ppm rel. to SiMe₄; coupling constants J in Hz.

### Example 1: Photooxidation of Citronellol in a Vortex Flow Reactor With TPP in Toluene

For construction and use of a vortex flow reactor see M. Poliakoff, Michael W. George et al. Org. Process Res. Dev. 21, 1042-1050 (2017).

One chiller for the external jacket of the vortex reactor was set to 20° C. and two chillers used for the 3 LEDs block (white light) were set to 15° C.).

In a 200 mL glass bottle, citronellol (36.2 g, 0. 23 mol) was mixed with toluene (58 mL), biphenyl as internal standard (2.9 g, 18.6 mmol) and tetraphenylporphyrin (TPP) (143 mg, 0.23 mmol). The 2.3 M citronellol solution was sonificated in an ultrasonic bath for 5 minutes at room temperature in the dark. Afterwards, the solution was pumped through the bottom pump (inlet pump) to introduce the mixture into the vortex reactor at a flow rate of 4 mL/min. The Vortex rotor was switched on and the rotation speed was allowed to increase smoothly to 4,000 rpm. The top pump (outlet pump) was programmed at 400 mL(min (to allow the gas to exit in order to avoid pressurisation of the reactor). The oxygen flow rate was setup at 240 mL/min-using the gas flow controller. The home-made water-cooled LED blocks were switched on setting the light intensity to the maximum, corresponding to 72000 lumens of white light generated by phosphor converted white LED's. After 13 minutes (time needed to equilibrate the flow reactor), the crude peroxides in toluene were collected for 4.5 minutes, corresponding to a volume of 18 mL of solution (collected in a measuring cylinder). The reaction was stopped.

In a round bottle flask of 100 mL, Na₂SO₃ (8.76 g, 69.5 mmol) was dissolved in deionised water (35 mL). The collected crude peroxide mixture was given to the aqueous quenching solution of Na₂SO₃. The biphasic mixture was stirred vigorously at room temperature for 24 hours. Complete quenching was confirmed using a peroxide strip.

The biphasic crude mixture was separated, the organic layer was washed with brine and the combined aqueous phases were extracted with toluene. The combined organic phases were dried over anhydrous Na₂SO₄ and concentrated in vacuo giving a purple organic crude (7.9 g). GC-FID analysis showed three major peaks (two diols and biphenyl). The crude was purified by flash chromatography (20% ethyl acetate in pentane) to remove the internal standard giving (E)-3,7-dimethyloct-5-ene-1,7-diol and 3,7-dimethyloct-7-ene-1,6-diol (7.3 g, 42.2 mmol, yield 91%) as a clear oil as confirmed by GC-FID analysis and 1H NMR analysis which is consistent with the literature.
Regioisomer ratio: 1:1
Conversion (%): >99
Productivity (mmol/min): 9.2 +/−0.5

### Example 2:

Following the general procedure described in Example 1 under the conditions provided in Table 1 the photooxidation of citronellol was repeated.

TABLE 1

| | Flow rate | TPP | | | |
|---|---|---|---|---|---|
| | substrate in | loading | parameters | Conversion | Productivity |
| Entry | solvent/O2 | (mol %) | changed | (%)^{a} | (mmol/min) |
| 1 | 4/240 | 0.2 | TPP loading | >99 | 9.2 +/− 0.5 |
| 2 | 5/292 | 0.2 | flow rates | 93% +/− 5 | 10.7 +/− 0.6 |
| 3 | 5/292 | 0.3 | TPP loading | 93% +/− 5 | 11.0 +/− 0.6 |
| 4 | 4/240 | 0.1 | 0° C. | >98 | 9.2 +/− 0.5 |
| 5 | 4/240 | 0.1 | 10° C. | >98 | 9.2 +/− 0.5 |
| 6 | 4/240 | 0.1 | 30° C. | >99 | 9.2 +/− 0.5 |
| 7 | 4/240 | 0.1 | 40° C. | >99 | 9.2 +/− 0.5 |

Conditions: light intensity 72000 lumens of white light generated by phosphor converted white LED's. a) Conversion calculated from the crude product with internal standard by 1H-NMR, results confirmed by GC-FID analysis.

### Example 3: Photooxidation of Ethylcitronellol in a Vortex Flow Reactor With TPP in Toluene

Ethycitronellol (3,7-dimethylnon-6-en-1-ol; E/Z ratio about 3:2) (36.4 g, 0.21mol) was mixed with toluene (58 mL), biphenyl as internal standard (3.4 g, 21.9 mmol) and tetraphenylporphyrin (131 mg, 0.21 mmol) and the 2.1 M ethyl citronellol solution was photooxidized as described in Example 1. The crude peroxides in toluene were collected for 5 minutes, giving a volume of 20 mL of solution. After quenching and work-up as described in example 1 a purple organic crude (7.32 g) was obtained showing three diols and biphenyl by GC-FID analysis. After flash chromatography (E)-3,7-dimethylnon-5-ene-1,7-diol (dr ˜1:1) and 3-methyl-7-methylenenonane-1,6-diol (dr ˜1:1) were obtained (6.65 g, 35.7 mmol, 85% yield) with a regioisomer ratio of 1.3:1 according to NMR and GC. Conversion: >99%. Productivity: 9.2+/−0.5 mmol/min. The product mixture was analyzed by GCMS and NMR (600 MHZ), the latter comprising HSQC-DEPT, HMBC, COSY and NOESY experiments.

Analytical data of (5E)-3,7-dimethylnon-5-ene-1,7-diol (mixture of two major diastereomers, roughly 1:1): ¹H NMR (C₆D₆) δ: 5.64-5.56 (m, 2H), 5.44 (d, J=15.4 Hz, 1H), 5.43 (d, J=15.4 Hz, 1H), 3.56-3.43 (m, 4H), 2.02-1.95 (m, 2H), 1.91-1.85 (m, 2H), 1.68-1.43 (m, 8H), 1.28-1.21 (m, 2H), 1.19 (2s, 6H), 0.90 (2t, J=7.5 Hz, 6H), 0.68 (2d, J=6.6 Hz, 6H) ppm. ¹³C NMR (C₆D₆) δ: 139.0 (d), 138.9 (d), 126.2 (d), 126.1 (d), 72.7 (2s), 60.6 (2t), 40.2 (t), 40.1 (t), 39.5 (t), 39.4 (t), 35.7 (t), 35.6 (t), 30.0 (2d), 28.0 (q), 27.8 (q), 19.8 (2q), 8.6 (2q) ppm. GCMS (t_{R} 7.33, two diastereomers under one peak): m/z =186 [M] + (0.2%), 171 [M-15] + (2%), 157 (4%), 139 (42%), 121 (22%), 99 (10%), 97 (10%), 95 (31%), 83 (11%), 81 (29%), 71 (31%), 69 (35%), 57 (26%), 55 (29%), 43 (100%), 41 (23%), 31 (31%), 29 (19%), 27 (13%).

Analytical data of 3-methyl-7-methylidenenonane-1,6-diol (mixture of two minor diastereomers, roughly 1:1): ¹H NMR (C₆D₆) δ: 5.07 (s, 1H), 5.06 (s, 1H), 4.85-4.83 (m, 2H), 3.98-3.92 (m, 2H), 3.56-3.43 (m, 4H), 2.13-2.05 (m, 2H), 1.95-1.88 (m, 2H), 1.63 -1.46 (m, 9H), 1.42-1.36 (m, 1H), 1.32-1.24 (m, 3H), 1.13-1.07 (m, 1H), 1.03 (2t, J=7.5 Hz, 6H), 0.85 (2d, J=6.6 Hz, 6H) ppm. ¹³C NMR (C₆D₆) δ: 154.2 (s), 154.1 (s), 108.2 (t), 108.8 (t), 75.9 (d), 75.3 (d), 60.6 (2t), 40.0 (2t), 33.3 (t), 33.2 (t) 33.0 (t), 32.8 (t), 29.8 (d), 29.3 (d), 24.2 (t), 24.0 (t), 20.0 (q), 19.9 (q), 12.5 (q), 12.4 (q) ppm. GCMS (t_{R} 7.69, two diastereomers under one peak): m/z=157 (3%), 139 (10%), 113 (8%), 100 (7%), 86 (17%), 85 (100%), 69 (27%), 55 (33%), 41 (32%), 29 (28%).

### Example 4: Photooxidation of Citronellol With TPP in Toluene in Batch

A red-violett 1.3 M solution of Citronellol (3 g, 18.24 mmol) and meso-tetraphenylporphyrine (12 mg, 18 μmol) in toluene (10 ml) is irradiated with white LED light and with a continuous oxygen stream bubbled through the reaction mixture at 40° C.

After 24 h and complete conversion detected by GC the violett solution is poured onto a stirred solution of Na₂SO₃ (3.5 g, 27.4 mmol) in water and is stirred for 22 h. After phase separation the organic layer is washed with saturated NaCl. The combined water layers (pH 7-8) are extracted with toluene. The organic layers are combined, dried over MgSO₄, filtered and evaporated giving a violettish oil (3.1 g) which is purified by flash chromatography through a 50 g SiO₂ cartridge using a 10-100% gradient of tert-butyl methyl ether in heptane giving 2.9 g (86%) of a mixture of (E)-3,7-dimethyloct-5-ene-1,7-diol and 3,7-dimethyloct-7-ene-1,6-diol (ratio 52:48). The analytical data are consistent with the literature.

### Example 5: Photooxidation of Citronellol With TPP in Toluene in Batch With Air

A solution of Citronellol (3 g, 18.2 mmol) and meso-tetraphenylporphyrine (12 mg, 18 μmol) in toluene (10 ml) is irradiated with a 300 W Osram Ultra Vitalux lamp and a continuous stream of compressed air is bubbled through the reaction mixture at 25-55° C. After 41 h and complete conversion detected by GC the violett solution is poured onto a stirred solution of Na₂SO₃ (3.5 g, 27.4 mmol) in water (20 ml) and is stirred for 23 h. After phase separation the organic phase is washed with saturated NaCl (30 ml). The combined water layers (pH 7-8) are with toluene (30 ml). The organic layers are combined, dried over MgSO₄, filtered and evaporated giving a violett oil (2.9 g) which is purified by flash chromatography through a 50 g SiO2 cartridge using a 10-100% gradient of tert-butyl methyl ether in heptane giving 2.42 g (77%) of a mixture of (E)-3,7-dimethyloct-5-ene-1,7-diol and 3,7-dimethyloct-7-ene-1,6-diol (ratio 52:48). Flash chromatography partially separated both isomers. The analytical data are consistent with the ones from example 4 and from the literature.

### Example 6: Photooxidation of Citronellol With TPP in Toluene Followed by Destruction of the Peroxides and Cyclization to Rose Oxide

The chillers used for the reaction were allowed to reach the desired temperature (one chiller for the external jacket of the vortex reactor was set to 20° C. and two chillers used for the LEDs block were set to 15° C.).

In a 200 mL glass bottle, Citronellol (50.3 g, 322 mmol) was mixed with toluene (81 mL) and tetraphenylporphyrin (0.2 g, 0.32 mmol). The 2.3 M solution was sonicated in an ultrasonic bath for 5 minutes at room temperature in the dark. Afterwards, the solution was pumped using the bottom pump (inlet pump) to introduce the mixture to the vortex reactor at 4 ml/min. The rotation speed of the Vortex reactor was allowed to increase to 4000 rpm. The top pump (outlet pump) was programmed at 400 ml/min (to allow the gas to exit and to avoid pressurisation of the reactor). The oxygen flow rate was set to 292 ml/min using the gas flow controller. The LED blocks were switched on.

After 10 minutes the reaction crude was collected for 20 minutes, which correspond to a volume of 80 ml of crude. The crude was collected in a round bottom flask filled with the quenching solution, Na₂SO₃ (34 g, 270 mmol) dissolved in water (130 ml) and toluene (50 ml) was added in order to dilute the crude solution pumped out of the reactor). The biphasic mixture was stirred vigorously at room temperature for 24 hours to reduce the peroxides (complete quench was confirmed using peroxide strip band test). The biphasic crude mixture was separated and the aqueous phase extracted once with toluene (25 mL) giving 144 g of combined organic layers as purple solution containing the diol isomers.

In a round bottom flask, Aliquat 336 (417 mg, 1 mmol) and 50% H₂SO₄ (20 g, 102 mmol) were mixed unter magnetic stirring. 103 ml of the he diol solution in toluene obtained above was added dropwise over a period of 15 min. The mixture was stirred at ambient temperature for 20 h. Complete conversion of the diols was checked by GC-FID, the biphasic phase was separated and the organic phase washed with 3 M NaOH an then with water to pH 7. The combined aqueous phase were extracted once with toluene (10 mL). The combined organic phase was dried over anhydrous Na₂SO₄ and concentrated in vacuo affording a purple organic crude (13.6 g) which was purified by flash chromatography (0-10% diethyl ether in pentane) affording cis-rose oxide (7.1 g 42% yield) with a purity of 97% (measured by ¹H NMR analysis using biphenyl as standard).

### Example 7: Photooxidation of Ethylcitronellol With TPP in Toluene Followed by Destruction of the Peroxides and Cyclization to Ethyl Rose Oxide (4-methyl-2-(2-methylbut-1-en-1-yl)tetrahydro-2H-pyran)

The chillers used for the reaction were allowed to reach the desired temperature (one chiller for the external jacket of the vortex reactor was set to 20° C. and two chillers used for the LEDs block were set to 15° C.).

In a 200 mL glass bottle, ethylcitronellol (36 g, 209 mmol) was mixed with toluene (58 mL) and tetraphenylporphyrin (130 mg, 0.21 mmol). The 2.1 M solution was sonicated in an ultrasonic bath for 5 minutes at room temperature in the dark. Afterwards, the solution was pumped using the bottom pump (inlet pump) to introduce the mixture to the vortex reactor at 4 ml/min. The rotation speed of the Vortex reactor was allowed to increase to 4000 rpm. The top pump (outlet pump) was programmed at 400 ml/min (to allow the gas to exit and to avoid pressurisation of the reactor). The oxygen flow rate was set to 292 ml/min using the gas flow controller. The LED blocks were switched on.

After 10 minutes the reaction crude was collected for 10 minutes, which correspond to a volume of 40 ml of crude. The crude was collected in a round bottom flask filled with the quenching solution, Na₂SO₃ (20 g, 159 mmol) dissolved in water (80 ml) and toluene (30 ml) was added to dilute the crude solution pumped out of the reactor. The biphasic mixture was stirred vigorously at room temperature for 24 hours to reduce the peroxides (complete quench was confirmed using peroxide strip band test).

The biphasic crude mixture was separated and the aqueous phase extracted once with toluene (15 mL) giving 88 ml of combined organic layers as a purple solution containing the diol isomers.

In a round bottom flask Aliquat 336 (115 mg, 0.28 mmol), 50% H₂SO₄ (5.15 g, 26.3 mmol) were mixed under magnetic stilling. 28 ml of the diol solution in toluene obtained above was added dropwise over a period of 15 minutes. The reaction mixture was stirred at room temperature for 20 hours. Full conversion of the diols was analysed by GC-FID analysis and the biphasic mixture was separated. The organic phase was washed 3M NaOH and water to pH 7. The combined aqueous phase was extracted once with toluene (5 mL). The combined organic phase was dried over anhydrous Na₂SO₄, and concentrated in vacuo affording a purple organic crude (4.3 g) which was purified by flash chromatography (0-10% diethyl ether in pentane) affording cis-ethyl rose oxide (2.2g, 49% yield) with a purity of 92% (measured by ¹H NMR analysis using biphenyl as standard).

1. A method for making a mixture comprising a compound of Formula (II), a compound of Formula (III), and, if R¹ is not methyl, a compound of formula (V) 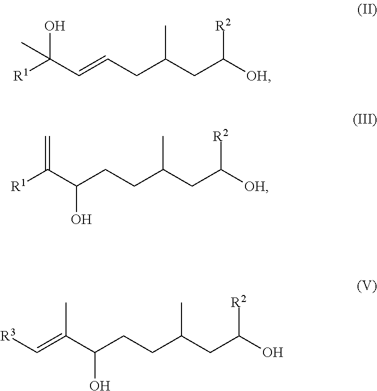
wherein the method comprises photooxidation of a compound of Formula (I) in an aromatic solvent comprising a sensitizer in the presence of molecular oxygen, 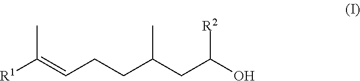
wherein
R¹ is selected from C₁-C₅ alkyl,
R² is selected from hydrogen and C₁-C₄ alkyl, and
R³ is selected from C₁-C₄ alkyl; and
wherein the sensitizer is a porphyrin.

2. The method of claim 1, wherein the sensitizer is selected from porphyrins of Formula (A) 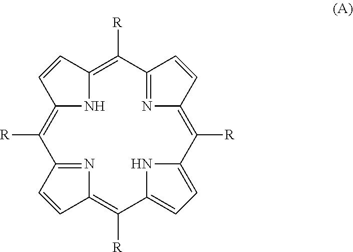
wherein R is selected from phenyl and substituted phenyl.

3. The method of claim 1, wherein the sensitizer is selected from porphyrin metal complex of Formula (B) 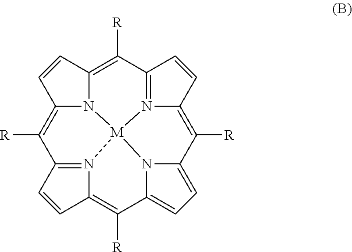
wherein M is a transition metal (II) or MX(III) wherein X is selected from halides and M is selected from Ga(III) Cl, Pd(II), and Zn(II), and
wherein R is selected from phenyl and substituted phenyl.

4. The method according to claim 2, wherein the sensitizer is selected from tetraphenylporphyrin, 5,10,15,20-(tetra-p-tolyl)porphyrin, 5,10,15,20-(tetra-4-chlorophenyl)porphyrin, 5,10,15,20-(tetra-4-trifluoromethylphenyl)-porphyrin, 5,10,15,20-(tetra-4-bromophenyl)porphyrin, 5,10,15,20-(tetra-2,6-difluorophenyl)porphyrin, 5,10,15,20-(tetra-2,6-chlorophenyl)porphyrin, and 5,10,15,20-(tetrapentafluorophenyl)porphyrin.

5. The method of claim 1, wherein the sensitizer is present at a concentration of 0.1 mol % or less.

6. The method of claim 1, wherein at least 0.5 M of the compound of Formula (I) is present.

7. The method of claim 6, wherein at least 1 M of the compound of Formula (I) is present.

8. The method of claim 1, wherein the method takes place in a flow reactor.

9. The method of claim 1, wherein the obtained mixture comprising the compound of Formula (II) and the compound of Formula (III) is cyclised to a compound of Formula (IV) in the presence of an acid 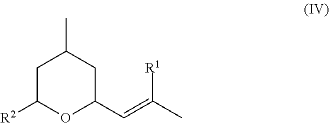
wherein R¹ is selected from C₁-C₅ alkyl, and
R² is selected from hydrogen and C₁-C₄ alkyl.

10. The method of claim 9, wherein the compound of Formula (IV) is 2-(2-methylprop-1-enyl)-4-methyltetrahydropyran or 4-methyl-2-(2-methylbut-1-en-1-yl)tetrahydro-2H-pyran.

11. The method of claim 8, wherein the compound of Formula (IV) is enriched in its cis-isomer.

12. The method of claim 9, wherein the compound of Formula (IV) is enriched in its cis-isomer.

13. The method of claim 1, wherein the aromatic solvent is selected from toluene, xylene, and chlorobenzene.
